(19) United States
(12) Patent Application Publication
(10) Pub. No.: US 2025/0177118 A1
(43) Pub. Date: Jun. 5, 2025

(54) DEVICES AND METHODS FOR BREAST TISSUE EXPANSION

(71) Applicant: THE METHODIST HOSPITAL, Houston, TX (US)

(72) Inventor: Aldona J. SPIEGEL, Houston, TX (US)

(21) Appl. No.: 18/842,267

(22) PCT Filed: Feb. 28, 2023
(86) PCT No.: PCT/US2023/014168

§ 371 Date: Aug. 28, 2024

Related U.S. Application Data
(60) Provisional application No. 63/314,858, filed on Feb. 28, 2022.

(51) Int. Cl. A61F 2/12

(52) U.S. Cl. CPC A61F 2/12; A61F 2250/0003

(57) ABSTRACT

Various implementations include a breast tissue expander device. The device includes a body and one or more tabs for coupling the device to a patient. The body has a first surface defining a convex surface, a second surface, a circumferential edge defined along an abutment of the first and second surfaces, a superior end, and an inferior end. A portion of the body has a maximum thickness of the body as measured between the first and second surfaces and is closer to the inferior end than to the superior end. Each of the one or more tabs has a first side, a second side, a first edge, and a second edge. The first edge of each tab is coupled to the body. At least one tab is coupled to the inferior end of the body. The at least one tab defines a total of two or more suture openings.

### CROSS-REFERENCE TO RELATED APPLICATIONS

This application claims the benefit of priority to U.S. Provisional Application No. 63/314,858 filed Feb. 28, 2022, which is hereby incorporated herein by reference in its entirety.

### BACKGROUND

Breast tissue expansion is a common technique used for breast reconstruction. After a mastectomy, a breast tissue expander is disposed under the breast skin and muscle to be coupled to the chest tissue wall. Previous and current breast tissue expanders feature a few suture tabs extending from the body of the expander that are spaced circumferentially around the edge of the body of the expander for suturing the device to the chest tissue. Each of the tabs define a single suture opening for suturing the breast tissue expander to the chest wall. Once the expander is sutured into place, the breast tissue expander essentially “hangs” from the sutures, which can cause excessive strain on the portions of the chest tissue to which the device is coupled.

Previous breast tissue expanders included textured surfaces that acted similarly to hook-and-loop connectors with the adjacent tissue to hold the tissue expander in place. Because of the textured design of these previous breast tissue expanders, a minimal number of sutures were necessary, and thus provided, to secure the device in place. However, recent studies have found that the textured surfaces of the previous breast tissue expanders caused an increased risk of breast implant-associated anaplastic large cell lymphoma, leading to the recall and discontinuation of textured breast tissue expanders.

To address this increased risk of breast implant-associated anaplastic large cell lymphoma, current breast tissue expanders feature smooth surfaces. Because the smooth surfaces of the current breast tissue expanders no longer benefitted from the hook-and-loop-like coupling of the previous textured expanders, current breast tissue expanders have a higher risk of moving out of place in the breast, even when properly sutured. The “tear-drop” shape of expanders also causes the devices to be susceptible to rotating within the breast.

Another issue with both the previous and current breast tissue expanders is the surgeon's accessibility to the suture tabs during implantation. Current methods of mastectomy allow for smaller incisions, which leads to a less invasive procedure but provides limited access to the chest tissue wall for the surgeon. For example, nipple sparing mastectomies involve a small incision overlying the nipple that allows the surgeon to remove all of the glandular tissue from the breast. During such a surgery, one or more of the circumferentially spaced suture tabs may not be easily accessible to the surgeon.

Thus, there exists a need for a smooth surface breast tissue expander that can be securely coupled to the chest tissue wall by one or more easily accessible suture tabs without causing the expander to hang by the sutures from the chest tissue.

Another issue with current breast tissue expanders is their lack of ability to drain seroma from around the expander throughout the expansion process. Typically, a drain tube is disposed adjacent a breast tissue expander when the expander is initially implanted. The removal of tissue from the breast during a mastectomy can lead to seroma accumulation around the expander, which can be drained using the drain tube. The separate drain tube is eventually removed from the patient while the expander remains implanted. However, as the expander is expanded, any empty spaces in the breast around the expander can accumulate seroma.

Some current breast tissue expanders include perforated drains along the inferior end of the expander and aspiration ports that, similar to the expansion ports of the expanders, allow access to the drain to aspirate the seroma adjacent the drain. However, these drains can become clogged and are not always located where pockets of seroma may form.

Thus, there exists a need for a breast tissue expander that has the ability to better drain seroma around the expander without becoming clogged.

### SUMMARY

Some of the aspects of the present disclosure relate to a breast tissue expander device, the device comprising: a body having a first surface, a second surface opposite the first surface, a circumferential edge of the body defined along an abutment of the first surface and the second surface, a superior end, and an inferior end opposite the superior end; the first surface defines a convex surface, wherein a portion of the body has a maximum thickness of the body as measured from the first surface to the second surface, and the portion of the body is closer to the inferior end than to the superior end; and one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the tabs is coupled to the inferior end of the body, wherein the at least one of the tabs defines a total of two or more suture openings extending from the respective first side to the respective second side.

In some aspects, in addition, or in the alternative, to any preceding aspects, all of the suture openings of the device are defined by the at least one of the tabs.

In some aspects, in addition, or in the alternative, to any preceding aspects, the one or more tabs comprises one tab.

In some aspects, in addition, or in the alternative, to any preceding aspects, the at least one of the tabs is coupled to the circumferential edge.

In some aspects, in addition, or in the alternative, to any preceding aspects, the at least one of the tabs extends along at least 75% of the inferior end of the body.

In some aspects, in addition, or in the alternative, to any preceding aspects, the at least one of the tabs extends along 70 mm-100 mm of the inferior end of the body.

In some aspects, in addition, or in the alternative, to any preceding aspects, the one of the at least one tabs includes two or more suture openings.

In some aspects, in addition, or in the alternative, to any preceding aspects, each of the two or more suture openings are spaced 2 mm-10 mm apart from each other.

In some aspects, in addition, or in the alternative, to any preceding aspects, the each of the two or more suture openings are spaced 4 mm-6 mm apart from each other.

In some aspects, in addition, or in the alternative, to any preceding aspects, the two or more suture openings comprises four or more suture openings.

In some aspects, in addition, or in the alternative, to any preceding aspects, the device further comprises a drain tube.

In some aspects, in addition, or in the alternative, to any preceding aspects, the drain tube is a Blake drain.

In some aspects, in addition, or in the alternative, to any preceding aspects, a center line of the drain tube extends substantially parallel to the circumferential edge along at least the inferior end of the body.

In some aspects, in addition, or in the alternative, to any preceding aspects, the at least one of the tabs is coupled to the inferior end of the body, the at least one of the tabs is coupled to a portion of the body that is closer than the drain tube to the circumferential edge.

In some aspects, in addition, or in the alternative, to any preceding aspects, the at least one of the tabs is coupled to the circumferential edge.

In some aspects, in addition, or in the alternative, to any preceding aspects, the second edge of the at least one of the tabs extends at least 10 mm from the drain tube.

In some aspects, in addition, or in the alternative, to any preceding aspects, the inferior end of the body includes an inframammary ridge portion, the drain tube extends along the inframammary ridge portion.

In some aspects, in addition, or in the alternative, to any preceding aspects, a center line of the drain tube extends around at least 50% of the circumferential edge.

In some aspects, in addition, or in the alternative, to any preceding aspects, the center line of the drain tube extends around at least 75% of the circumferential edge.

In some aspects, in addition, or in the alternative, to any preceding aspects, the device is a smooth surface tissue expander (STE).

Some of the aspects of the present disclosure relate to a breast tissue expander device, the device comprising: a body having a first surface, a second surface opposite the first surface, a circumferential edge of the body defined along an abutment of the first surface and the second surface, a superior end, and an inferior end opposite the superior end, the first surface defining a convex surface, wherein a portion of the body has a maximum thickness of the body as measured from the first surface to the second surface, and the portion of the body is closer to the inferior end than to the superior end; and one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the one or more tabs defines two or more suture openings extending from the first side to the second side.

In some aspects, in addition, or in the alternative, to any preceding aspects, the at least one of the tabs is coupled to the inferior end of the body.

In some aspects, in addition, or in the alternative, to any preceding aspects, all of the suture openings of the device are defined by the at least one of the tabs.

In some aspects, in addition, or in the alternative, to any preceding aspects, the one or more tabs comprises one tab.

In some aspects, in addition, or in the alternative, to any preceding aspects, the at least one of the tabs is coupled to the circumferential edge.

In some aspects, in addition, or in the alternative, to any preceding aspects, the at least one of the tabs extends along at least 75% of the inferior end of the body.

In some aspects, in addition, or in the alternative, to any preceding aspects, the at least one of the tabs extends along 70 mm-100 mm of the inferior end of the body.

In some aspects, in addition, or in the alternative, to any preceding aspects, each of the two or more suture openings are spaced 2 mm-10 mm apart from each other.

In some aspects, in addition, or in the alternative, to any preceding aspects, each of the two or more suture openings are spaced 4 mm-6 mm apart from each other.

In some aspects, in addition, or in the alternative, to any preceding aspects, the two or more suture openings comprises four or more suture openings.

In some aspects, in addition, or in the alternative, to any preceding aspects, further comprising a drain tube.

In some aspects, in addition, or in the alternative, to any preceding aspects, the drain tube is a Blake drain.

In some aspects, in addition, or in the alternative, to any preceding aspects, a center line of the drain tube extends substantially parallel to the circumferential edge along at least the inferior end of the body.

In some aspects, in addition, or in the alternative, to any preceding aspects, the at least one of the tabs is coupled to the inferior end of the body, the at least one of the tabs is coupled to a portion of the body that is closer than the drain tube to the circumferential edge.

In some aspects, in addition, or in the alternative, to any preceding aspects, the at least one of the tabs is coupled to the circumferential edge.

In some aspects, in addition, or in the alternative, to any preceding aspects, the second edge of the at least one of the tabs extends at least 10 mm from the drain tube.

In some aspects, in addition, or in the alternative, to any preceding aspects, the inferior end of the body includes an inframammary ridge portion, the drain tube extends along the inframammary ridge portion.

In some aspects, in addition, or in the alternative, to any preceding aspects, a center line of the drain tube extends around at least 50% of the circumferential edge.

In some aspects, in addition, or in the alternative, to any preceding aspects, the center line of the drain tube extends around at least 75% of the circumferential edge.

In some aspects, in addition, or in the alternative, to any preceding aspects, the device is a smooth surface tissue expander (STE).

Some of the aspects of the present disclosure relate to a breast tissue expander device, the device comprising: a body having a first surface, a second surface opposite the first surface, a circumferential edge of the body defined along an abutment of the first surface and the second surface, a superior end, and an inferior end opposite the superior end, the first surface defining a convex surface, wherein a portion of the body has a maximum thickness of the body as measured from the first surface to the second surface, and the portion of the body is closer to the inferior end than to the superior end; and a drain tube coupled to the body and extending substantially parallel to the circumferential edge, wherein a center line of the drain tube extends around at least 50% of the circumferential edge of the body.

In some aspects, in addition, or in the alternative, to any preceding aspects, the center line of the drain tube extends around at least 75% of the circumferential edge.

In some aspects, in addition, or in the alternative, to any preceding aspects, the drain tube extends along the entire inferior end of the body.

In some aspects, in addition, or in the alternative, to any preceding aspects, the drain tube is a Blake drain.

In some aspects, in addition, or in the alternative, to any preceding aspects, the body of the device defines an aspiration port in fluid communication with at least one end of the drain tube.

In some aspects, in addition, or in the alternative, to any preceding aspects, the aspiration port is in fluid communication with two ends of the drain tube.

In some aspects, in addition, or in the alternative, to any preceding aspects, the inferior end of the body includes an inframammary ridge portion, and the drain tube extends along the inframammary ridge portion.

In some aspects, in addition, or in the alternative, to any preceding aspects, further comprising one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the tabs is coupled to the inferior end of the body, wherein the at least one of the tabs defines a total of two or more suture openings extending from the respective first side to the respective second side.

In some aspects, in addition, or in the alternative, to any preceding aspects, all of the suture openings of the device are defined by the at least one of the tabs.

In some aspects, in addition, or in the alternative, to any preceding aspects, the one or more tabs comprises one tab.

In some aspects, in addition, or in the alternative, to any preceding aspects, the at least one of the tabs is coupled to the circumferential edge.

In some aspects, in addition, or in the alternative, to any preceding aspects, the at least one of the tabs extends along at least 75% of the inferior end of the body.

In some aspects, in addition, or in the alternative, to any preceding aspects, the at least one of the tabs extends along 70 mm-100 mm of the inferior end of the body.

In some aspects, in addition, or in the alternative, to any preceding aspects, one of the at least one tabs includes two or more suture openings.

In some aspects, in addition, or in the alternative, to any preceding aspects, each of the two or more suture openings are spaced 2 mm-10 mm apart from each other.

In some aspects, in addition, or in the alternative, to any preceding aspects, each of the two or more suture openings are spaced 4 mm-6 mm apart from each other.

In some aspects, in addition, or in the alternative, to any preceding aspects, the two or more suture openings comprises four or more suture openings.

In some aspects, in addition, or in the alternative, to any preceding aspects, the at least one of the tabs is coupled to the inferior end of the body, and the at least one of the tabs is coupled to a portion of the body that is closer than the drain tube to the circumferential edge.

In some aspects, in addition, or in the alternative, to any preceding aspects, the at least one of the tabs is coupled to the circumferential edge.

In some aspects, in addition, or in the alternative, to any preceding aspects, the second edge of the at least one of the tabs extends at least 10 mm from the drain tube.

In some aspects, in addition, or in the alternative, to any preceding aspects, the device is a smooth surface tissue expander (STE).

### BRIEF DESCRIPTION OF THE DRAWINGS

Example features and implementations are disclosed in the accompanying drawings. However, the present disclosure is not limited to the precise arrangements and instrumentalities shown.

FIG. 1 shows a top view of an example breast tissue expander device, according to one implementation.

FIG. 2 shows a cross-sectional view of the breast tissue expander device shown in FIG. 1 taken along line 2-2.

FIG. 3 shows a top view of an example breast tissue expander device, according to another implementation.

FIG. 4 shows a top view of an example breast tissue expander device, according to another implementation.

FIG. 5 shows an end view of a drain tube, according to one implementation.

FIG. 6 shows a top view of an example breast tissue expander device, according to another implementation.

FIG. 7 shows a top view of an example breast tissue expander device, according to another implementation.

FIG. 8 shows a top view of an example breast tissue expander device, according to another implementation.

### DETAILED DESCRIPTION

The devices, systems, and methods disclosed herein provide for a breast tissue expander device with a suture tab extending from the inferior end of the body of the device. The tab can be elongated and extend along most of the inferior end of the body. The tab of the devices disclosed herein define a plurality of suture openings aligned parallel to the inferior end of the body such that a surgeon can tie a suture in a baseball stitch through the openings to couple the tab of the device to the inframammary ridge of the patient. The location of the tab along the inferior end of the body of the device provides a “shelf”' that allows the body to be supported from below the body by the coupled tab rather than being hung from one or more suture tabs coupled along the superior end or the sides of the body like with current breast tissue expander devices.

The devices, systems, and methods disclosed herein further provide for a breast tissue expander device with one or more suture tabs coupled at any location and each defining two or more suture openings.

The devices, systems, and methods disclosed herein further provide for a breast tissue expander device with an integrated drain tube that extends around the circumferential edge of the expander. Because the drain extends around the circumferential edge of the expander, rather than only the inferior end as with some current expanders, the drain is able to access seroma pockets at any location around the expander throughout the entire expansion process. As seroma forms in the breast, the expander can displace any seroma that has accumulated in the inferior portion of the breast, causing the seroma to migrate superiorly. Also, pockets of seroma can form and become trapped in other locations around the expander other than the inferior portion of the breast. The circumferentially extending integrated drain tubes disclosed in the devices, systems, and methods herein are able to access this fluid such that the fluid can be aspirated through an integrated port of the expander.

Furthermore, the drain tubes disclosed in some implementations are Blake drains that define a series of axially extending channels spaced apart from each other circumferentially. These channels provide more opening area than the perforated drains of existing expanders to prevent clogging of the openings. Also, with current expander drains located at the inferior end of the expander, the weight of the expander causes the inferior end of the expander to press against the tissue at the inferior end of the breast, clogging the drain openings. Because the drain tubes disclosed herein are extending along the circumferential edge of the expanders, the abutment of the expander against the inferior tissue of the breast does not block the only openings of the drain tube.

The drain tubes are fluidically coupled to an aspiration port that can be accessed through the overlying tissue of the breast by piercing the tissue and port with a syringe needle. The syringe can then be used to cause a suction force to extract the seroma through the drain tube, through the aspiration port, and into the syringe.

Various implementations include a breast tissue expander device. The device includes a body and one or more tabs for coupling the device to a patient. The body has a first surface, a second surface opposite the first surface, a circumferential edge of the body defined along an abutment of the first surface and the second surface, a superior end, and an inferior end opposite the superior end. The first surface defines a convex surface. A portion of the body has a maximum thickness of the body as measured from the first surface to the second surface, and the portion of the body is closer to the inferior end than to the superior end. Each of the one or more tabs has a first side, a second side opposite from the first side, a first edge extending from the first side to the second side, and a second edge opposite from the first edge. The first edge of each of the one or more tabs is coupled to the body. At least one of the tabs is coupled to the inferior end of the body. The at least one of the tabs defines a total of two or more suture openings extending from the respective first side to the respective second side.

Various other implementations include a breast tissue expander device. The device includes a body and one or more tabs for coupling the device to a patient. The body has a first surface, a second surface opposite the first surface, a circumferential edge of the body defined along an abutment of the first surface and the second surface, a superior end, and an inferior end opposite the superior end. The first surface defines a convex surface. A portion of the body has a maximum thickness of the body as measured from the first surface to the second surface, and the portion of the body is closer to the inferior end than to the superior end. Each of the one or more tabs has a first side, a second side opposite from the first side, a first edge extending from the first side to the second side, and a second edge opposite from the first edge. The first edge of each of the one or more tabs is coupled to the body. At least one of the one or more tabs defines two or more suture openings extending from the first side to the second side.

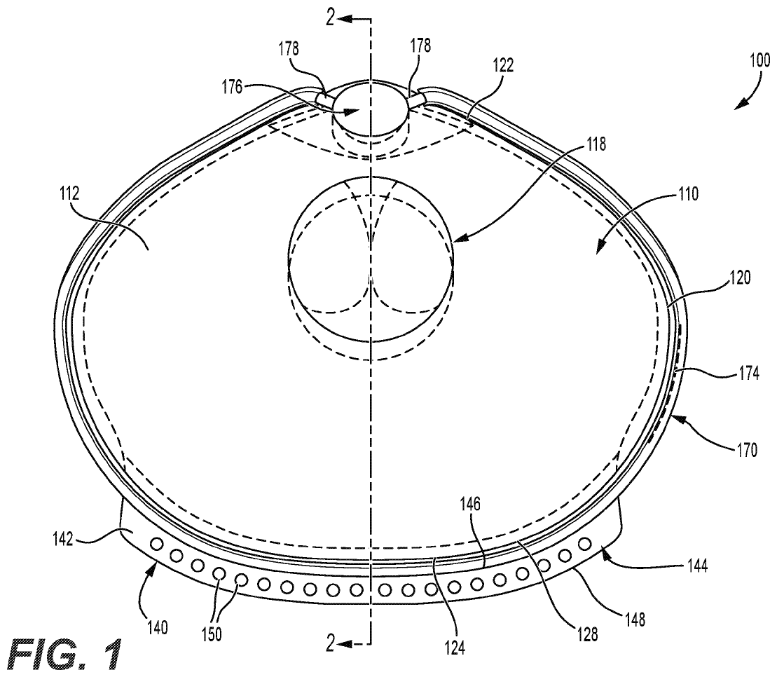

FIGS. 1-4 show breast tissue expander devices 100, 200, 300, according to various aspects. The devices 100, 200, 300 each include a body 110, 210, 310, at least one tab 140, 240, 340, and a drain tube 170, 270, 370.

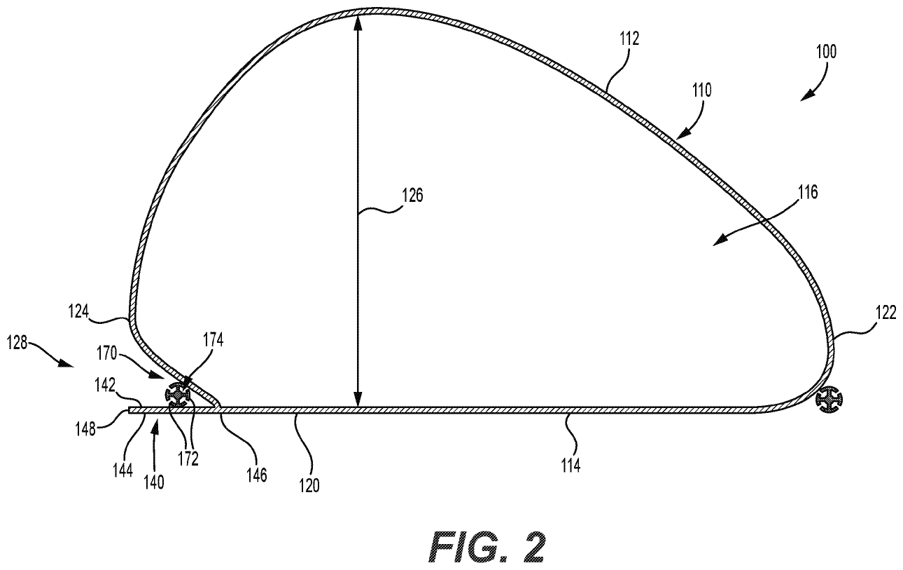

FIGS. 1 and 2 show a first implementation of a breast tissue expander device 100. The body 110 of the device 100 shown in FIGS. 1 and 2 has a first surface 112 and a second surface 114 opposite the first surface 112. The first surface 112 defines a convex surface configured to face away from a patient, and the second surface 114 is configured to abut the chest tissue of the patient. The first surface 112 and the second surface 114 of the body 110 define a cavity 116 that can be filled with sterile saltwater to cause expansion of the body 110. The body 110 (i.e., first surface 112 and second surface 114) is a smooth surface tissue expander (STE).

The first surface 112 of the body 110 defines an expansion port 118. The expansion port 118 can be accessed using a syringe and needle, or any other pumping device, that can cause sterile saltwater to flow into the cavity 116 of the body 110 to expand the body 110. Although the expansion port 118 shown in FIG. 1 is an integrated port disposed within the cavity 116 and accessible along the first surface 112, in some implementations, the expansion port is a top port disposed external to the first surface.

The body 110 further has a circumferential edge 120 defined along an abutment of the first surface 112 and the second surface 114, a superior end 122, and an inferior end 124 opposite the superior end 122. The body 110 is a “teardrop” shape such that a portion of the body 110 that is closer to the inferior end 124 than to the superior end 122 has the maximum thickness 126 of the body 110 as measured from the first surface 112 to the second surface 114. The inferior end 124 of the body 110 includes an inframammary ridge portion 128 configured to be located adjacent the inframammary ridge of the patient when the device 100 is coupled to the chest of the patient. The body 110 of the device 100 can be sized according to the desired expansion of the tissue.

The body 110 of the device 100 shown in FIGS. 1 and 2 is made of silicone, but in other implementations, the body can be made of any other material suitable for implantation in the body of a patient.

The device 100 shown in FIGS. 1 and 2 includes a single tab 140 used for coupling the device 100 to the chest of a patient. The tab 140 has a first side 142, a second side 144 opposite from the first side 142, a first edge 146 extending from the first side 142 to the second side 144, and a second edge 148 opposite from the first edge 146. The first edge 146 of the tab 140 is coupled to the body 110 along the inferior end 124 of the body 110. The tab 140 shown in FIGS. 1 and 2 is coupled to the body 110 of the device 100 by being integrally formed with the body 110, but in other implementations, the tab is separately formed from the body of the device and is coupled by known means to the body. The tab 140 shown in FIGS. 1 and 2 is made of the same material as the body 110, but in implementations in which the tab is a separately formed from the body and coupled to the body, the tab can be made of silicone or any other material suitable for implantation in the body of a patient.

The tab 140 shown in FIGS. 1 and 2 is coupled to the circumferential edge 120 of the body 110 along the inferior end 124. The tab 140 defines a total of twenty suture openings 150 extending from the first side 142 of the tab 140 to the second side 144 of the tab 140. The tab 140 shown in FIGS. 1 and 2 extends along 75% of the inferior end 124 of the body 110, but in other implementations, the tab extends more or less than 75% of the inferior end of the body. In some implementations, the tab extends along 70 mm-100 mm of the inferior end of the body, depending on the size and shape of the body. The location of the tab 140 along the inferior end 124 of the body 110 allows the body 110 to be attached to the inframammary ridge of the patient. The inframammary ridge contains enough tissue to provide a secure coupling point for the device 100 and allows the device 100 to naturally rest on the inframammary ridge.

Each of the suture openings 150 shown in FIGS. 1 and 2 is spaced 5 mm apart from the adjacent suture openings 150 and/or the edges of the tab 140. The spacing of the suture openings 150 provide enough attachment points for a surgeon to tie a baseball stitch suture along each tab 140 to securely couple the tab 140 to the chest tissue of a patient. However, in some implementations, each suture opening is spaced apart from the adjacent suture openings and/or the edges of the tab by 4 mm-6 mm. In some implementations, each suture opening is spaced apart from the adjacent suture openings and/or the edges of the tab by 2 mm-10 mm.

Although the tab 140 shown in FIGS. 1 and 2 defines a total of twenty suture openings 150, in other implementations, the tab defines any number of two or more suture openings extending from the first side to the second side of the tab. In some implementations, the two or more suture openings includes four or more suture openings to provide enough attachment points to securely couple the device to the chest tissue of a patient.

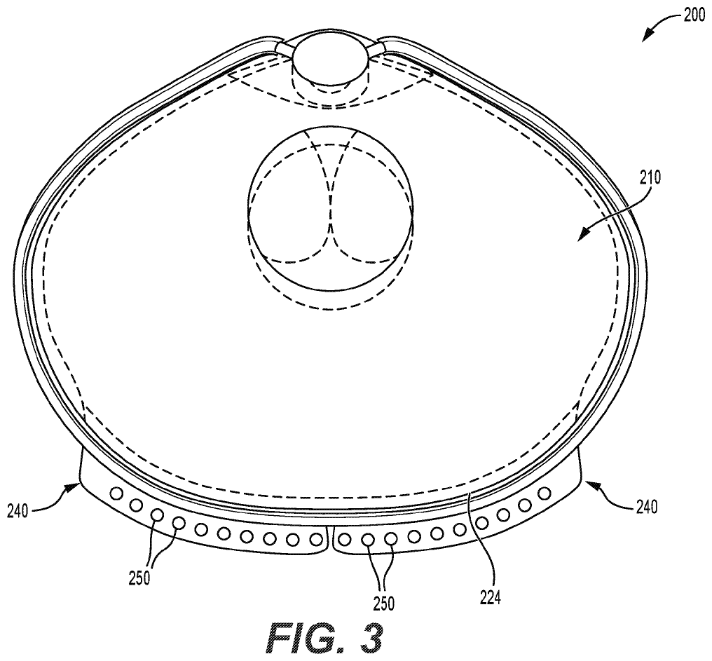

In some implementations, such as the implementation shown in FIG. 3, the device 200 includes two tabs 240 coupled to the inferior end 224 of the body 210. The device 200 shown in FIG. 3 is similar to the device 100 shown in FIGS. 1 and 2, so reference numbers are used in FIG. 3 that are similar to those used in FIGS. 1 and 2 to describe similar features. Each of the tabs 240 includes nine suture openings 250 to provide for a total of twenty suture openings 250, similar to the device 100 shown in FIGS. 1 and 2. Although the implementation of the device 200 shown in FIG. 3 includes two tabs 240, in other implementations, the device can include any number of tabs coupled to the inferior edge and each of the tabs can include any number of openings. For example, in some implementations, the device could include Y number of tabs coupled to the inferior end of the body, and each tab could define Z suture openings such that the Y number of tabs define a total of Y×Z suture openings. In some implementations, the device can further include any number of tabs coupled to any other portion of the body as long as at least one tab is coupled to the inferior edge of the body and the at least one tab defines a total of 2 suture openings.

The drain 170 shown in FIGS. 1 and 2 is a Blake drain defining four channels 172 or groves extending longitudinally along the drain 170. Each of the four channels 172 is spaced apart circumferentially from the other channels by 90 degrees. The drain 170 allows seroma accumulating around the device 100 to be evacuated using a suction source. The drain tube 170 includes a center line 174 extending along its longitudinal axes. The center line 174 of the drain tube 170 shown in FIGS. 1 and 2 extends around, and substantially parallel to, the entire circumferential edge 120 of the body 110, but in other implementations, the center line of the drain tube extends at least 75% of the circumferential edge. In some implementations, the center line of the drain tube extends around at least 50% of the circumferential edge. In some implementations, the center line of the drain tube along at least the inferior end of the body.

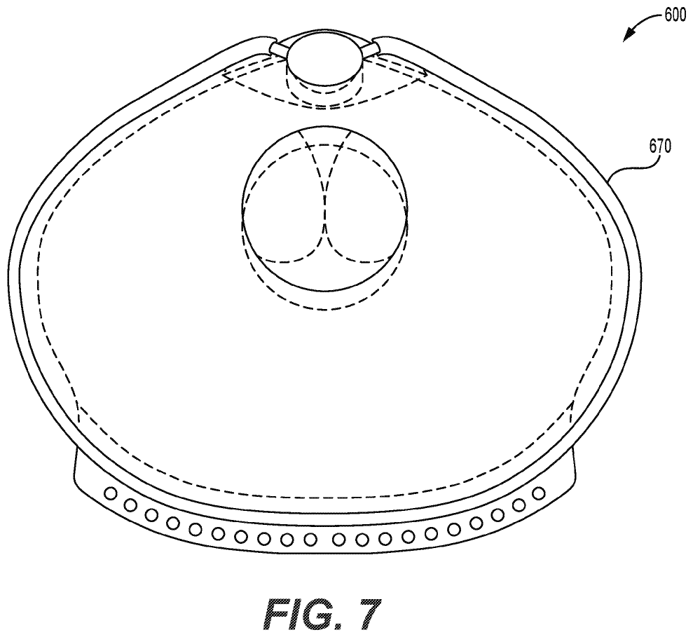

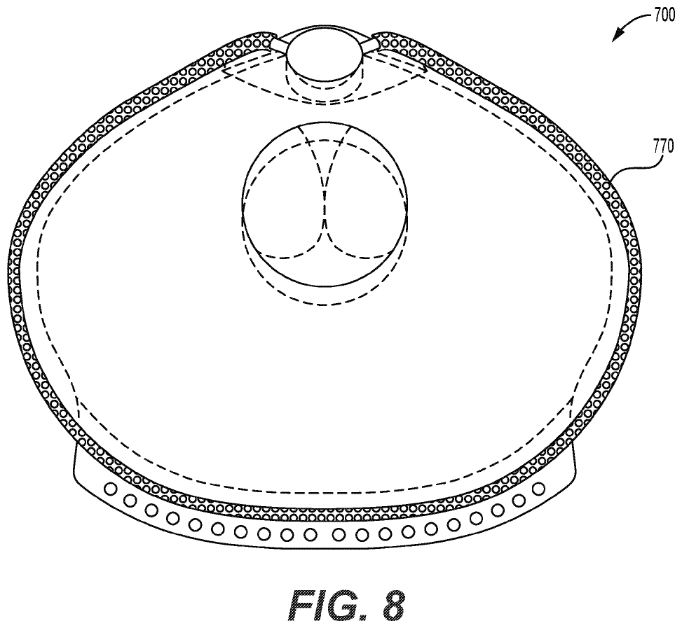

The drain tube 170 shown in FIGS. 1 and 2 is a Blake drain, but in other implementations, the drain tube can be any other type of drain known in the art. For example, FIG. 7 shows a device 600 that includes a solid tube drain 670, and FIG. 8 shows a device 700 that includes a perforated tube drain 770.

The axial ends of the drain tube 170 are coupled to a Y-connector 178 such that the drain tube 170 is in fluid communication with an aspiration port 176. The aspiration port 176 can be accessed using a syringe and needle, or any other suction device, that can cause a negative pressure within the drain tube 170 to cause the seroma accumulated around the device 100 to flow through the channels 172 of the drain tube 170, through the aspiration port 176, and out of the breast. Although the aspiration port 176 shown in FIG. 1 is an integrated port disposed within the cavity 116 and accessible along the first surface 112, in some implementations, the aspiration port is a top port disposed external to the first surface.

It is also contemplated that the drain tube 170, or any other drain tubes disclosed herein, could be used for injecting treatment such as chemotherapy or antibiotics in addition to or in replacement of evacuation of seroma.

The drain tube 170 shown in FIGS. 1 and 2 extends along the inframammary ridge portion 128 of the body 110 such that the tab 140 is coupled to a portion of the body 110 that is closer than the drain tube 170 to the circumferential edge 120. Thus, the tab 140 can be coupled to the chest tissue of a patient by sutures such that the tab 140 is flat against the chest tissue and the drain 170 extends over the tab 140. The second edge 148 of the tab 140 can extend beyond the drain tube 170 by at least 10 mm.

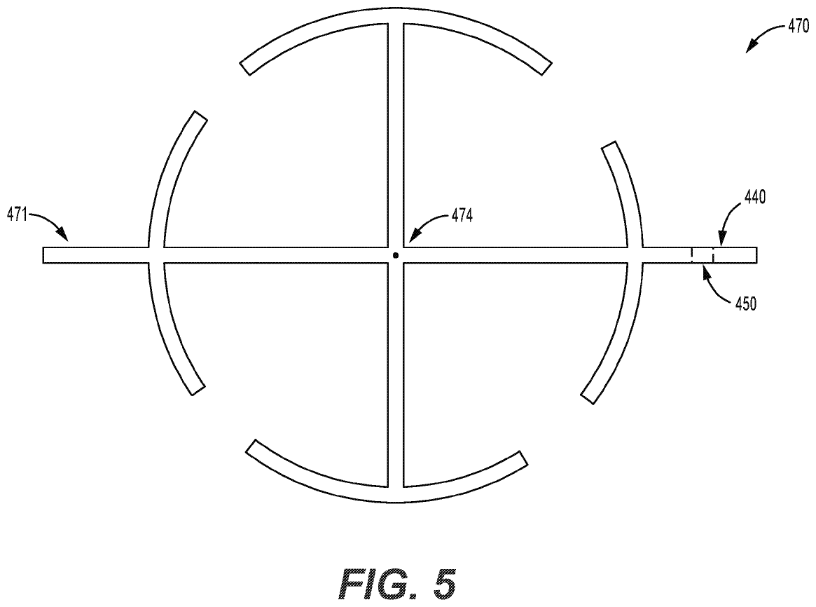

FIG. 5 shows another implementation of a drain tube 470. The drain tube shown in FIG. 5 is a Blake drain similar to the drain tube 170 shown in FIGS. 1 and 2. However, the drain tube 470 shown in FIG. 5 includes one or more tabs 440 defining one or more suture openings 450 and one or more coupling flanges 471. The one or more coupling flanges 471 can be coupled to a body of a device to secure the drain tube 470 to the body. Once the coupling flanges are coupled to the body of a device, the one or more tabs 440 can be used to couple the device to the chest tissue of a patient. Although the present application is directed to breast tissue expander devices, it is contemplated that the drain tube 470 shown in FIG. 5 could be used with any implantable device.

The one or more tabs 440 extend radially outwardly from an outer surface of the drain tube 470. The one or more coupling flanges 471 extend radially outwardly from the outer surface of the drain tube 470 such that the one or more coupling flanges 471 are diametrically opposed from the one or more tabs 440. However, in some implementations, the one or more coupling flanges 471 extend radially outwardly from the outer surface of the drain tube 470 such that the one or more coupling flanges 471 are transverse to the one or more tabs 440.

The one or more tabs 440 can be axially spaced apart from the one or more coupling flanges 471, or one or more of the tabs 440 can be disposed directing across the center line 474 from one of the coupling flanges 471.

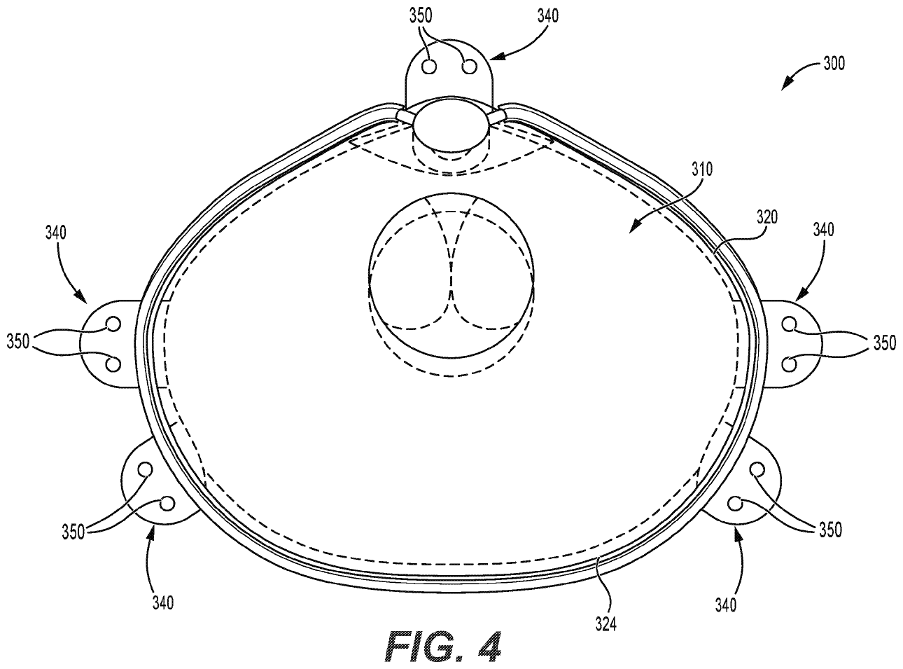

FIG. 4 shows another breast tissue expander device 300, according to another implementation. The device 300 shown in FIG. 4 is similar to the device 100 shown in FIGS. 1 and 2, so reference numbers are used in FIG. 4 that are similar to those used in FIGS. 1 and 2 to describe similar features. The device 300 shown in FIG. 4 has a body 310 similar to the device 100 shown in FIGS. 1-3. However, the tabs 340 of the device 300 shown in FIG. 4 are not coupled to the inferior end 324 of the body 310. Instead, the tabs 340 are coupled to the body 310 such that the tabs 340 are spaced around the circumferential edge 320.

Each of the tabs 340 includes two or more suture openings 350. Existing breast tissue expanders include tabs that each define only a single suture opening for coupling the device to the chest of the patient. The single suture allows the device to pivot about the suture out of position, and if the single suture fails, then the device can become decoupled from the patient's chest tissue. The two or more suture openings 350 of the tabs 340 of the device 300 shown in FIG. 4 provide enough attachment points for a surgeon to tie a baseball stitch suture along each tab 340 to securely couple each of the tabs 340 to the chest tissue of a patient.

The device 300 shown in FIG. 4 includes five tabs 340, but in other implementations, the device can include any number of tabs. In some implementations, each tab can include any number of suture openings, as long as at least one of the tabs defines two or more suture openings. The tabs 340 shown in FIG. 4 are spaced around the circumferential edge 320 of the body 310, but in other implementations, the tabs are coupled to the body at strategic locations to provide more secure coupling points of the device to the chest tissue of a patient. In some implementations, at least one of the tabs is located along the inferior end of the body of the device.

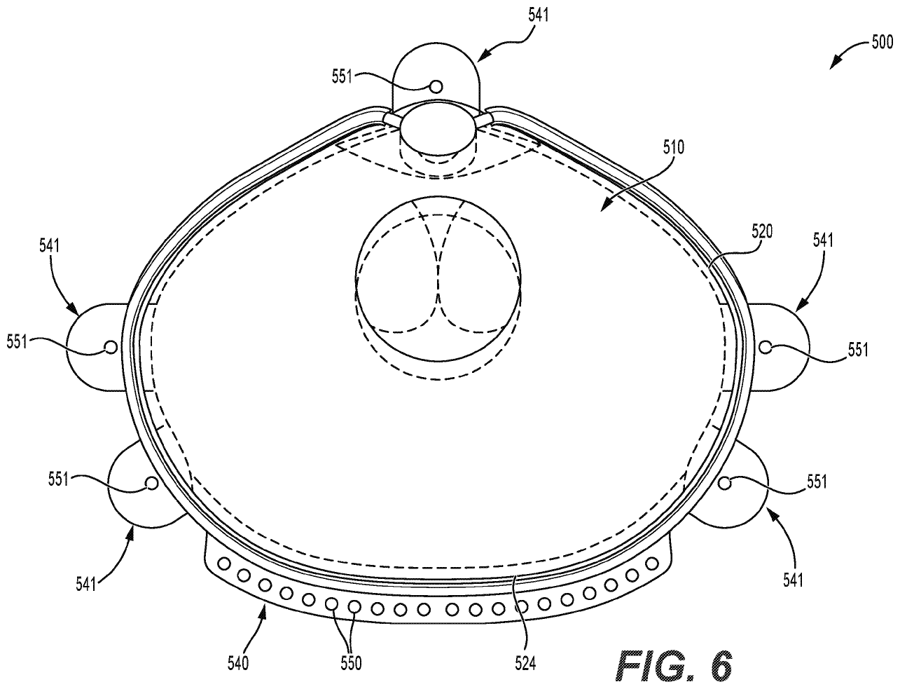

FIG. 6 shows another breast tissue expander device 500, according to another implementation. The device 500 shown in FIG. 6 is similar to the device 100 shown in FIGS. 1 and 2, so reference numbers are used in FIG. 6 that are similar to those used in FIGS. 1 and 2 to describe similar features. The device 500 shown in FIG. 6 has a body 510 similar to the device 100 shown in FIGS. 1-4. However, the device 500 shown in FIG. 6 includes an elongated tab 540 coupled to the inferior end 524 of the body 510, similar to the device 100 shown in FIGS. 1 and 2, and five tabs 541 are coupled to the body 510 such that the tabs 541 are spaced around the circumferential edge 520.

Each of the tabs 541 includes one or more suture openings 551. The suture openings 551 of the elongated tab 540 can be used to anchor the device to the chest tissue of the patient to form a supporting “shelf,” similar to the device 100 shown in FIGS. 1 and 2, while the suture openings 551 of the tabs 541 can be used to keep the device 500 in the correct orientation.

Although the tab 540 of the device shown in FIG. 6 includes twenty suture openings 550, as with the device 100 of FIGS. 1 and 2, in some implementations, the device can include any number of suture openings located at any spacing relative to each other or relative to the ends of the tab. In some implementations, the device includes two or more tabs coupled to the inferior end, similar to the device 200 shown in FIG. 3, along with the tabs spaced around the circumferential edge.

The device 500 shown in FIG. 6 includes five tabs 541, but in other implementations, the device can include any number of tabs. In some implementations, each tab can include any number of suture openings. The tabs 541 shown in FIG. 6 are spaced around the circumferential edge 520 of the body 510, but in other implementations, the tabs are coupled to the body at strategic locations to provide more secure coupling points of the device to the chest tissue of a patient.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A breast tissue expander device, the device comprising:
a body having a first surface, a second surface opposite the first surface, a circumferential edge of the body defined along an abutment of the first surface and the second surface, a superior end, and an inferior end opposite the superior end, the first surface defining a convex surface, wherein a portion of the body has a maximum thickness of the body as measured from the first surface to the second surface, and the portion of the body is closer to the inferior end than to the superior end; and
one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the tabs is coupled to the inferior end of the body, wherein the at least one of the tabs defines a total of two or more suture openings extending from the respective first side to the respective second side.

2. The device of claim 1, wherein all of the suture openings of the device are defined by the at least one of the tabs.

3. The device of claim 1, wherein the one or more tabs comprises one tab.

4. The device of claim 1, wherein the at least one of the tabs is coupled to the circumferential edge.

5. The device of claim 1, wherein the at least one of the tabs extends along at least 75% of the inferior end of the body.

6. The device of claim 1, wherein the at least one of the tabs extends along 70 mm-100 mm of the inferior end of the body.

7. The device of claim 1, wherein one of the at least one tabs includes two or more suture openings.

8. The device of claim 1, wherein each of the two or more suture openings are spaced 2 mm-10 mm apart from each other.

9. The device of claim 8, wherein each of the two or more suture openings are spaced 4 mm-6 mm apart from each other.

10. The device of claim 1, wherein the two or more suture openings comprises four or more suture openings.

11. The device of claim 1, further comprising a drain tube.

12. The device of claim 11, wherein the drain tube is a Blake drain.

13. The device of claim 11, wherein a center line of the drain tube extends substantially parallel to the circumferential edge along at least the inferior end of the body.

14. The device of claim 13, wherein the at least one of the tabs is coupled to the inferior end of the body, wherein the at least one of the tabs is coupled to a portion of the body that is closer than the drain tube to the circumferential edge.

15. The device of claim 14, wherein the at least one of the tabs is coupled to the circumferential edge.

16. The device of claim 14, wherein the second edge of the at least one of the tabs extends at least 10 mm from the drain tube.

17. The device of claim 11, wherein the inferior end of the body includes an inframammary ridge portion, wherein the drain tube extends along the inframammary ridge portion.

18. The device of claim 11, wherein a center line of the drain tube extends around at least 50% of the circumferential edge.

19. The device of claim 18, wherein the center line of the drain tube extends around at least 75% of the circumferential edge.

20. The device of claim 1, wherein the device is a smooth surface tissue expander (STE).

21. A breast tissue expander device, the device comprising:
a body having a first surface, a second surface opposite the first surface, a circumferential edge of the body defined along an abutment of the first surface and the second surface, a superior end, and an inferior end opposite the superior end, the first surface defining a convex surface, wherein a portion of the body has a maximum thickness of the body as measured from the first surface to the second surface, and the portion of the body is closer to the inferior end than to the superior end; and
one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the one or more tabs defines two or more suture openings extending from the first side to the second side.

22. The device of claim 21, wherein the at least one of the tabs is coupled to the inferior end of the body.

23. The device of claim 22, wherein all of the suture openings of the device are defined by the at least one of the tabs.

24. The device of claim 21, wherein the one or more tabs comprises one tab.

25. The device of claim 22, wherein the at least one of the tabs is coupled to the circumferential edge.

26. The device of claim 22, wherein the at least one of the tabs extends along at least 75% of the inferior end of the body.

27. The device of claim 21, wherein the at least one of the tabs extends along 70 mm-100 mm of the inferior end of the body.

28. The device of claim 21, wherein each of the two or more suture openings are spaced 2 mm-10 mm apart from each other.

29. The device of claim 28, wherein each of the two or more suture openings are spaced 4 mm-6 mm apart from each other.

30. The device of claim 21, wherein the two or more suture openings comprises four or more suture openings.

31. The device of claim 21, further comprising a drain tube.

32. The device of claim 31, wherein the drain tube is a Blake drain.

33. The device of claim 31, wherein a center line of the drain tube extends substantially parallel to the circumferential edge along at least the inferior end of the body.

34. The device of claim 33, wherein the at least one of the tabs is coupled to the inferior end of the body, wherein the at least one of the tabs is coupled to a portion of the body that is closer than the drain tube to the circumferential edge.

35. The device of claim 34, wherein the at least one of the tabs is coupled to the circumferential edge.

36. The device of claim 34, wherein the second edge of the at least one of the tabs extends at least 10 mm from the drain tube.

37. The device of claim 31, wherein the inferior end of the body includes an inframammary ridge portion, wherein the drain tube extends along the inframammary ridge portion.

38. The device of claim 31, wherein a center line of the drain tube extends around at least 50% of the circumferential edge.

39. The device of claim 38, wherein the center line of the drain tube extends around at least 75% of the circumferential edge.

40. The device of claim 21, wherein the device is a smooth surface tissue expander (STE).

41. A breast tissue expander device, the device comprising:
a body having a first surface, a second surface opposite the first surface, a circumferential edge of the body defined along an abutment of the first surface and the second surface, a superior end, and an inferior end opposite the superior end, the first surface defining a convex surface, wherein a portion of the body has a maximum thickness of the body as measured from the first surface to the second surface, and the portion of the body is closer to the inferior end than to the superior end; and
a drain tube coupled to the body and extending substantially parallel to the circumferential edge, wherein a center line of the drain tube extends around at least 50% of the circumferential edge of the body.

42. The device of claim 41, wherein the center line of the drain tube extends around at least 75% of the circumferential edge.

43. The device of claim 41, wherein the drain tube extends along the entire inferior end of the body.

44. The device of claim 41, wherein the drain tube is a Blake drain.

45. The device of claim 41, wherein the body of the device defines an aspiration port in fluid communication with at least one end of the drain tube.

46. The device of claim 45, wherein the aspiration port is in fluid communication with two ends of the drain tube.

47. The device of claim 41, wherein the inferior end of the body includes an inframammary ridge portion, wherein the drain tube extends along the inframammary ridge portion.

48. The device of claim 41, further comprising one or more tabs for coupling the device to a patient, each of the one or more tabs having a first side, a second side opposite from the first side, a first edge extending from the first side to the second side, and a second edge opposite from the first edge, wherein the first edge of each of the one or more tabs is coupled to the body, wherein at least one of the tabs is coupled to the inferior end of the body, wherein the at least one of the tabs defines a total of two or more suture openings extending from the respective first side to the respective second side.

49. The device of claim 48, wherein all of the suture openings of the device are defined by the at least one of the tabs.

50. The device of claim 48, wherein the one or more tabs comprises one tab.

51. The device of claim 48, wherein the at least one of the tabs is coupled to the circumferential edge.

52. The device of claim 48, wherein the at least one of the tabs extends along at least 75% of the inferior end of the body.

53. The device of claim 48, wherein the at least one of the tabs extends along 70 mm-100 mm of the inferior end of the body.

54. The device of claim 48, wherein one of the at least one tabs includes two or more suture openings.

55. The device of claim 48, wherein each of the two or more suture openings are spaced 2 mm-10 mm apart from each other.

56. The device of claim 55, wherein each of the two or more suture openings are spaced 4 mm-6 mm apart from each other.

57. The device of claim 48, wherein the two or more suture openings comprises four or more suture openings.

58. The device of claim 48, wherein the at least one of the tabs is coupled to the inferior end of the body, wherein the at least one of the tabs is coupled to a portion of the body that is closer than the drain tube to the circumferential edge.

59. The device of claim 48, wherein the at least one of the tabs is coupled to the circumferential edge.

60. The device of claim 48, wherein the second edge of the at least one of the tabs extends at least 10 mm from the drain tube.

61. The device of claim 41, wherein the device is a smooth surface tissue expander (STE).
